(19) United States
(12) Patent Application Publication
(10) Pub. No.: US 2024/0326036 A1
(43) Pub. Date: Oct. 3, 2024

(54) APPARATUS FOR GAS SAMPLE COLLECTION

(71) Applicant: THE UNIVERSITY OF BRITISH COLUMBIA, Vancouver (CA)

(72) Inventors: Jane Emily HILL, Delta (CA); Miza MWANZA, Vancouver (CA); Ali Reza BAHARI, Vancouver (CA)

(21) Appl. No.: 18/735,207

(22) Filed: Jun. 6, 2024

Related U.S. Application Data
(63) Continuation of application No. PCT/CA2022/000062, filed on Dec. 6, 2022.
(60) Provisional application No. 63/286,232, filed on Dec. 6, 2021.

(51) Int. Cl. B01L 3/00

(52) U.S. Cl. CPC B01L 3/50; B01L 2200/026; B01L 2200/0689; B01L 2200/146; B01L 2300/021; B01L 2300/0627

(57) ABSTRACT

An apparatus for collecting a gas sample may include a cartridge fluidly connected to the gas sample at a first end of the cartridge, the cartridge having: one or more bores; and sample media in the one or more bores, and a pressure source capable of drawing the gas sample through the apparatus. In some embodiments, the apparatus may further comprise: a conduit with a first end fluidly connected to the gas sample and a second end opposite to the first end connected to the cartridge. The conduit further comprises an interface that fluidly connects the gas sample to the first end of the conduit, and one or more sensors capable of measuring one or more parameters of the gas sample within the conduit.

### REFERENCE TO RELATED APPLICATIONS

This application is a continuation of Patent Cooperation Treaty (PCT) application No. PCT/CA2022/000062 having an international filing date of 6 Dec. 2022, which in turn claims priority from, and for the purposes of the United States of America the benefit under 35 USC 119 in relation to, U.S. Patent Application No. 63/286,232 filed 6 Dec. 2021. All of the applications referred to in this paragraph are hereby incorporated herein by reference.

### TECHNICAL FIELD

This invention relates to the field of gaseous sample collection, in particular to apparatus for collecting breath samples and methods for operating same.

### BACKGROUND

There is a general desire to capture analytes from gaseous samples.

In some organisms, exhaled breath can contain a significant amount of information regarding the health of the subject. In humans, for example, the presence of acetone in the breath of a diabetic patient can indicate if a diabetic patient is experiencing ketosis. By way of further example, the detection of alcohol in the exhaled breath of an individual can indicate whether an individual is intoxicated.

There is also a growing body of scientific literature indicating that breath from humans contains various biomarkers which can be used to diagnose a number of diseases, including respiratory infections (tuberculosis, for example), cancers (breast cancer, for example), metabolic disorders (diabetes, for example), and gastrointestinal diseases (inflammatory bowel disease, for example). Information obtained from breath from humans can also be used to monitor general health and wellbeing, as well as monitoring for the presence of drugs (both legal and illegal).

Often, the diagnosis of many diseases requires the collection of an invasive sample (such as a blood sample, tissue biopsy, and bronchoalveolar lavage fluids). Not only is the collection of invasive samples unpleasant for the test subject, but it is also expensive and leads to an increased risk of infection and/or contamination. The collection of a non-invasive breath sample, on the other hand, is much more pleasant for the test subject, and less complex to collect than invasive samples.

Despite the advantages associated with collecting breath samples from test subjects, there remains a dearth of reliable apparatus available for collecting breath samples.

Furthermore, some prior art breath sampling devices make use of a gas sampling bag to collect a breath sample, whereas some other prior art breath sampling devices make use of a glass bulb to collect a breath sample. The utility of these devices is constrained as the volume of gas that can be analyzed is limited by the capacity of the gas sampling bag or glass bulb.

Examples of some breath sampling devices are disclosed in the following patent applications: WO2020/263185, and WO2020/148030.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides an apparatus for collecting analyte from a gas sample. The apparatus comprises: a cartridge fluidly connectable to the gas sample, the cartridge shaped to define one or more bores configurable to be in fluid communication with the gas sample; and sample media comprising sorbent material insertable into the one or more bores. A pressure gradient draws at least a portion of the gas sample into the cartridge and into contact with the sample media.

The gas sample may comprise exhaled human breath. The pressure gradient may be created at least in part by an exhalation pressure associated with the human breath.

The gas sample may comprise one or more of: ambient air, outgas from a biological sample, outgas from soil, outgas from waste, or outgas from plants.

The apparatus may comprise a pump for creating, at least in part, the pressure gradient. The pump my act directly on the gas sample or on the at least a portion of the gas sample.

The pressure gradient may be created, at least in part, by a pressure associated with the gas sample.

The gas sample may be contained in an inflated gas sampling bag.

The cartridge may be hermetically sealable with a first cap at a first end of the cartridge, and a second cap at a second end of the cartridge opposite to the first end of the cartridge. The first cap and the second cap may comprise a first washer seal and a second washer seal. The first washer seal and second washer seal may be fabricated from an inert material.

The apparatus may comprise at least one detector coupled to the cartridge. The at least one detector may comprise one or more of: a temperature detector, a shock detector, and a geolocator module.

The sample media may comprise one or more of: a thermal desorption tube, GERSTEL Twister®, Solid-Phase Microextraction fibers, Thin-Film Solid-Phase Microextraction (TF-SPME), polydimethylsiloxane (PDMS) stir bars, Markes quartz wool, Markes glass wool, and HiSorb™ probes.

The apparatus may comprise an identifier on the cartridge. The identifier may comprise one or more of: an RFID tag, and a barcode.

The apparatus may comprise a conduit with a first end fluidly connectable to the gas sample and a second end opposite to the first end, the second end connectable to the cartridge to provide fluid communication between the gas sample and the cartridge through the conduit. The conduit may comprise one or more sensors capable of measuring or otherwise detecting one or more parameters of the gas sample within the conduit.

The second end of the conduit may be detachably connectable to the cartridge. The second end of the conduit may be threadably connectable to the cartridge.

The apparatus may comprise an interface at the first end for facilitating the connection between the first end and the gas sample. The interface may comprise one of: a mouthpiece, a straw, a mask for a human, a mask for an animal, and a connector for a tracheal tube.

The interface may be detachably connectable to one or more of: the gas sample and the conduit.

The conduit may comprise a filter located in a fluid passage of the conduit between the gas sample and the cartridge. The filter may be in a position in the fluid passage of the conduit closer to the first end of the conduit than a moisture trap. The filter may be removable from the apparatus.

The apparatus may comprise a fan in fluid communication with a fluid passage of the conduit between the gas sample and the cartridge.

The apparatus may comprise a heating element for providing heat in a fluid passage of the conduit between the gas sample and the cartridge.

The apparatus may comprise a moisture trap located to remove moisture from a fluid passage of the conduit between the gas sample and the cartridge. The moisture trap may be located in a position in the fluid passage of the conduit closer to the first end of the conduit than the one or more sensors.

The apparatus may comprise a display for outputting indicia corresponding to one or more parameters measured with the one or more sensors.

The apparatus may comprise a valve connected to the conduit convertible between a first configuration where gas is permitted to flow between the gas sample and the cartridge through the conduit and a second configuration where the gas sample is diverted before reaching the cartridge (or the sample media).

The apparatus may comprise a processor. The processor may be connected to receive one or more signals from the one or more sensors, may be configured to determine a portion of the gas sample to be sampled based at least in part on the one or more signals, and, may be connected to actuate the valve between the first configuration and the second configuration based at least in part on the determined portion of the gas. The processor may be further configured to actuate a pump to impose the pressure gradient when the valve is in the first configuration.

The portion of the gas sample to be sampled may be an alveolar portion of a human's exhaled breath. The processor may be configured to determine one or more thresholds associated with determining the alveolar portion in a calibration operation of the apparatus based at least in part on the human's exhaled breath. The one or more thresholds may comprise a concentration of CO2. The one or more thresholds may comprise a rate of change of concentration of CO2.

The apparatus may comprise a baffle located in a fluid passage of the conduit between the gas sample and the cartridge. The baffle may be shaped to direct fluid to a subset of the one or more of the bores in the cartridge. The baffle may be configurable between a first position that directs fluid to a first subset of the one or more bores in the cartridge, and a second position that directs fluid to a second subset of the one or more bores.

The apparatus may comprise a key mechanism configured (e.g. shaped and/or located) to orient the cartridge in a first orientation relative to the conduit, so that the baffle is located to direct gas from the gas sample to a first subset of the one or more of the bores in the cartridge. The key mechanism may be further configured (e.g. shaped and/or located) to orient the cartridge in a second orientation relative to the conduit, so that the baffle is located to direct gas from the gas sample to a second subset of the one or more bores in the cartridge, the second subset of bores different from the first subset of bores.

Cross sections of the conduit and the cartridge may be shaped to provide complementary polygonal shapes. The cross sections of the conduit and the cartridge may be alignable in a first relative orientation where the baffle is located to direct gas from the gas sample to a first subset of the one or more bores in the cartridge. The cross-sections of the conduit and the cartridge may be alignable in a second relative orientation where the baffle is located to direct gas from the gas sample to a second subset of the one or more bores in the cartridge, the second subset of bores different from the first subset of bores.

The baffle may be shaped and/or dimensioned to promote laminar flow of the gas sample through a fluid passage of the conduit between the gas sample and the cartridge.

The apparatus may be battery powered.

Data from the one or more sensors may be exportable to a data acquisition device. Data exported to the data acquisition device may be associated with a sample tag affixed to or otherwise connected to the cartridge. The sample tag may comprise an RFID tag or a barcode. The data obtained from analyzing the sample media in the cartridge may be associated with the sample tag.

One or more of the sensors may comprise a flow sensor for detecting a rate of flow of the gas from the gas sample to the cartridge. The apparatus may comprise an indicator that indicates whether a particular flow rate has been achieved based on an output from the flow sensor. The indicator may comprise one or more of: an LED, and a rotameter.

The apparatus may comprise at least one one way valve in the conduit and convertible between a first configuration where gas is permitted to flow between the gas sample and the cartridge through the conduit and a second configuration where gas from the gas sample is prevented from reaching the cartridge.

The apparatus may comprise a ventilation port connected to purge excess gas from the gas sample.

The apparatus may comprise: a conduit with a first end fluidly connectable to the gas sample and a second end opposite to the first end connectable to a three way valve; an interface that fluidly connects the gas sample to the first end of the conduit; an exhaust port fluidly connected to a first exit of the three way valve; a baffle fluidly connected to a second exit of the three way valve, the baffle being shaped to direct fluid to at least one of the one or more of the bores in the cartridge; and one or more sensors capable of measuring or otherwise detecting one or more parameters of the gas sample within the conduit.

The baffle may be at least one of: configurable between a first position that directs fluid to a first subset of the one or more bores in the cartridge, and a second position that directs fluid to a second subset of the one or more bores; and shaped to direct fluid to a selectable subset of the one or more of the bores in the cartridge.

The interface may be detachably connectable to one or more of: the gas sample and the first end of the conduit.

The conduit may comprise a filter located in a fluid passage of the conduit between the gas sample and the first ends of the conduit.

The apparatus may comprise a processor. The processor may be connected to receive one or more signals from the one or more sensors, may be configured to determine a portion of the gas sample to be sampled based at least in part on the one or more signals and may be connected to actuate a three-way valve between a first configuration where gas is permitted to flow between the gas sample and the cartridge through the conduit and a second configuration where gas from the gas sample is prevented from reaching the cartridge. The portion of the gas to be sampled may comprise an alveolar fraction of a human's exhaled breath.

The apparatus may comprise a fan in fluid communication with the exhaust port.

The apparatus may comprise a detent mechanism configured to retain the cartridge in a position relative to the conduit that aligns the baffle with one or more of the one or more bores in the cartridge. The detent mechanism may comprise a bistable cam mechanism with a spring biased step member that indexes along an intermittent stop.

Data from the one or more sensors may be exportable to a data acquisition device.

Another aspect of the invention provides a method of collecting analyte from a gas sample. The method comprises: drawing at least a portion of the gas sample through one or more bores in a cartridge with a pressure gradient wherein the bores are filled with sample media; and adsorbing analyte in the gas sample onto the sample media.

The method may comprise imposing the pressure gradient directly on the gas sample or on the at least a portion of the gas sample with a pump.

The method may comprise: attaching a first end of a conduit to the gas sample; attaching a second end of the conduit to the cartridge so as to provide fluid communication between the gas sample and the cartridge through the conduit; and measuring one or more parameters of the gas sample in the conduit with one or more sensors.

The method may comprise: analyzing at least the one or more parameters of the gas sample within the conduit with a processor to determine a portion of the gas sample to be captured; and actuating a valve between: a first configuration where gas is permitted to flow between the gas sample and the cartridge through the conduit; and a second configuration where gas is diverted before reaching the cartridge; such that the portion of the gas sample to be captured is permitted to flow between the gas sample and the cartridge through the conduit.

The method may comprise analyzing the one or more parameters of the gas sample to determine when a concentration of a compound has been reached. The method may comprise analyzing the one or more parameters of the gas sample to determine when a rate of change of concentration of a compound has been reached. The method may comprise actuating the valve from the first configuration to the second configuration when the gas sample to be captured has flowed through the sample media in the cartridge.

The method may comprise actuating the valve from the first configuration to the second configuration when one or more of the following are fulfilled: a rate of change of a compound is reached; a time interval has passed since the valve actuated from the second configuration to the first configuration; and when a volumetric flow rate is reached.

The method may comprise: sensing a flow rate of gas in the cartridge with one or more sensors; and indicating whether a particular flow rate has been achieved based at least on the measurement of the one or more sensors. Indicating whether the particular flow rate has been achieved may comprise illuminating an LED. Indicating whether the particular flow rate has been achieved may comprise a float balanced between a first mark and a second mark.

The method may comprise: attaching a first end of a conduit to the gas sample; attaching a second end of the conduit to a three way valve; attaching the cartridge to a first exit of the three way valve; attaching an exhaust port to a second exit of the three way valve; and measuring one or more parameters of the gas sample within the conduit with one or more sensors.

The method may comprise analyzing the one or more parameters of the gas sample within the conduit with a processor to determine a portion of the gas sample to be captured; and actuating the three way valve between: a first configuration where gas is permitted to flow between the gas sample and the cartridge through the conduit; and a second configuration where gas is permitted to flow through the exhaust port; such that the portion of the gas sample to be captured is permitted to flow between the gas sample and the cartridge through the conduit.

The method may comprise analyzing the one or more parameters of the gas sample to determine when a concentration of CO2 has been reached. The method may comprise analyzing the one or more parameters of the gas sample to determine when a rate of change of concentration of CO2 has been reached.

The method may comprise any features, combinations of features and/or sub-combinations of features described herein.

Other aspects of the invention provide an apparatus having any combination or sub combination of features or elements as described herein.

Other aspects of the invention provide a method comprising any step, act, combination of steps and/or acts or sub combination of steps and/or acts as described herein.

Other aspects of the invention provide a non-transitory machine readable medium storing instructions that when executed by a data processor cause the data processor to perform a control method for an apparatus as described herein.

In addition to the exemplary aspects and embodiments described above, further aspects and embodiments will become apparent by reference to the drawings and by study of the following detailed descriptions.

It is emphasized that the invention relates to all combinations of the above features, even if these are recited in different claims.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a gas sampling apparatus according to an example embodiment.

FIG. 2A-1, FIG. 2A-2, FIG. 2A-3, FIG. 2A-4 (collectively, FIG. 2A) illustrate cross-sections of cartridges according to a number of example embodiments.

FIG. 2B is an exploded plan view of a cartridge according to an example embodiment.

FIG. 2C is a cross section of a cap according to an example embodiment.

FIG. 2D is a schematic plan view of a washer seal according to an example embodiment.

FIG. 3 is schematic illustration of a gas sampling apparatus according to an example embodiment.

FIG. 4A is a flowchart of the inputs to and outputs from a processor in a gas sampling apparatus according to an example embodiment.

FIG. 4B is a flowchart illustrating a method for operating the FIG. 3 gas sampling apparatus according to an example embodiment.

FIG. 5A is a graph of CO₂ concentration, volume of gas collected, and flow rate of gas as a function of time in a gas sampling apparatus according to an example embodiment.

FIG. 5B is a flowchart of a method of capturing a portion of a gas sample according to an example embodiment.

FIG. 6A is a perspective view of a prototype gas sampling apparatus according an example embodiment.

FIG. 6B is a side view of a prototype gas sampling apparatus according to an example embodiment.

FIG. 6C is a side view of a cartridge according to an example embodiment.

FIG. 7A is a schematic of a connection interface between a conduit and a cartridge in a gas sampling apparatus according to an example embodiment.

FIG. 7B-1 and FIG. 7B-2 (collectively, FIG. 7B) are cross sections of a connection interface between a conduit and a cartridge in a gas sampling apparatus according to an example embodiment.

FIG. 7C is a schematic of a connection interface between a conduit and a cartridge in a gas sampling apparatus according to an example embodiment.

FIG. 7D-1 and FIG. 7D-2 (collectively FIG. 7D) are cross sections of a connection interface between a conduit and a cartridge in a gas sampling apparatus according to an example embodiment.

FIGS. 7E-1, 7E-2 and 7E-3 (collectively, FIG. 7E) schematically depict cross sections of a connection interface between a conduit and a cartridge with a key mechanism in a gas sampling apparatus according to an example embodiment.

FIGS. 7F-1, 7F-2 and 7F-3 (collectively, FIG. 7F) schematically depict cross sections of a connection interface between a conduit and a cartridge in a gas sampling apparatus according to an example embodiment.

FIG. 7G is a perspective view of a baffle according to an example embodiment.

FIG. 7H is a front and rear view of a baffle according to an example embodiment.

FIG. 7I is a side view of a baffle according to an example embodiment.

FIG. 7J is a cross sectional view of a baffle according to an example embodiment.

FIG. 8 is a gas sampling apparatus according to an example embodiment.

FIGS. 9A and 9B are sectional views of a gas sampling apparatus according to an example embodiment.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

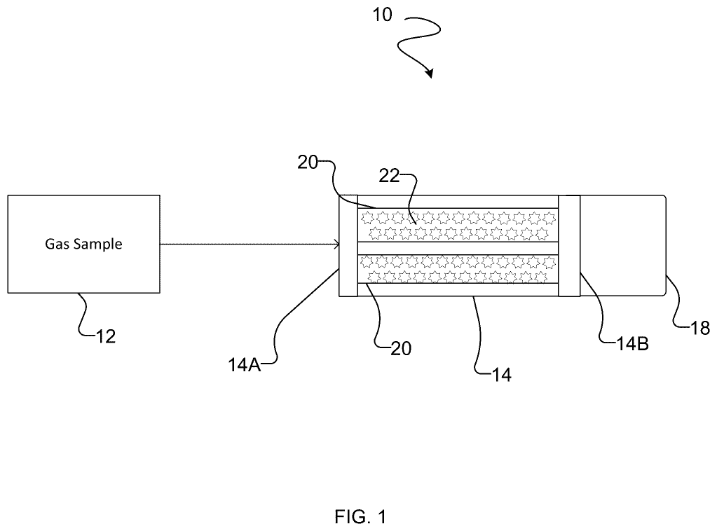

FIG. 1 is a gas sampling apparatus 10 according to an example embodiment. Gas sampling apparatus 10 comprises a cartridge 14 and a pressure source 18.

Cartridge 14 is shaped to define one or more bores 20 extending through the length of cartridge 14. Bores 20 are configured (e.g. by shape, size or otherwise) to accept sample media 22. In some embodiments, cartridge 14 is solid apart from bores 20. In other embodiments, cartridge 14 is all or partially hollow and bores 20 are provided in the form of one or more tubes within cartridge 14.

Sample media 22 may comprise any sorbent material capable of adsorbing an analyte from gas sample 12. In some embodiments, sample media 22 comprises a thermal desorption tube, but other additional or alternative sorbents can include: GERSTEL Twister®, Solid-Phase Microextraction fibers, Thin-Film Solid-Phase Microextraction (TF-SPME), polydimethylsiloxane (PDMS) stir bars, Markes quartz wool, Markes glass wool, HiSorb™ probes and/or the like.

In some embodiments, a pressure gradient in apparatus 10 (which is created when different regions of apparatus 10 have a different pressure) is created by a pressure source or is otherwise created to move gas within apparatus 10. In the illustrated embodiment of FIG. 1, pressure source 18 is fluidly connected to second end 14B of cartridge 14. In some embodiments, pressure source 18 comprises a pump. In some embodiments, pressure source 18 may be fluidly connected to first end 14A of cartridge 14 or elsewhere relative to cartridge 14. Although pressure source 18 is shown as an element of the FIG. 1 gas sampling apparatus 10, in some embodiments pressure source 18 may actually comprise or originate from gas sample 12 itself (i.e. gas sample 12 may be at a higher pressure than part(s) of (e.g. second end 14B of) cartridge 14, such that gas sample 12 is drawn through cartridge 14). For example, gas sample 12 could be gas contained within an inflated gas sampling bag (such as a Tedlar® bag), or pressure source 18 may be the exhalation pressure from exhaled breath.

One purpose of pressure source 18 is to assist with drawing gas sample 12 through cartridge 14; pressure source 18 acts on gas sample 12 itself to draw gas sample 12 through cartridge 14.

In some embodiments, gas sample 12 is exhaled human breath. Some humans (such as children) may have difficulty exhaling through sample media 22 at a desired flow rate. Consequently, pressure source 18 may be used to facilitate the flow of gas sample 12 to ensure that adequate adsorption onto sample media 22 is achieved. In some embodiments, gas sample 12 may comprise ambient air, in which case pressure source 18 may be used to draw the ambient air through cartridge 14.

Apparatus 10 of the FIG. 1 embodiment is operated by passing gas sample 12 through first end 14A of cartridge 14. Gas sample 12 then flows through bores 20. Analytes in gas sample 12 then adsorb onto sample media 22.

With the analytes of interest in gas sample 12 adsorbed onto sample media 22, cartridge 14 can be taken to an appropriate piece of testing equipment for analysis. Examples of appropriate testing equipment include, but are not limited to, mass spectrometers, and gas chromatographs.

As discussed above and elsewhere herein, in some embodiments gas sample 12 is human breath or ambient air. Gas sample 12 can also be outgas from various biological samples such as urine, blood, saliva, bronchoalveolar lavage fluid, stool and/or the like. Gas sample 12 could also be outgas from soil, waste, plants and/or the like. These are non-limiting examples of gas sample 12.

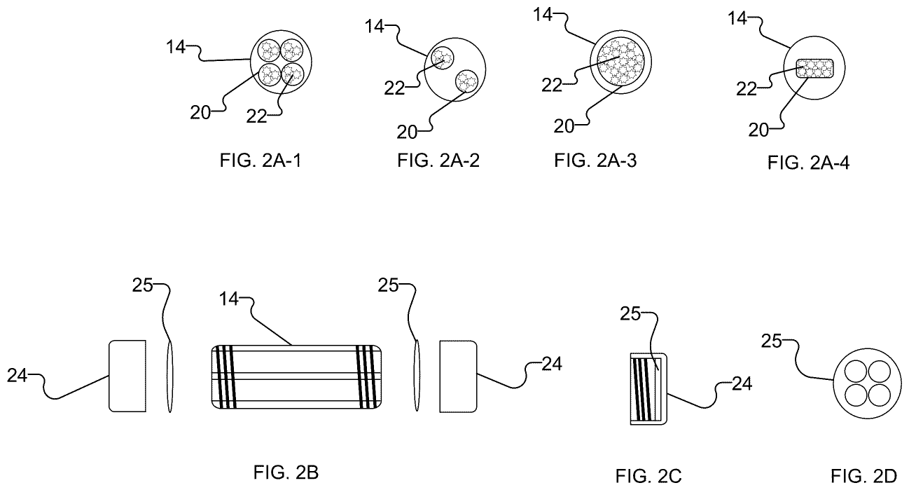

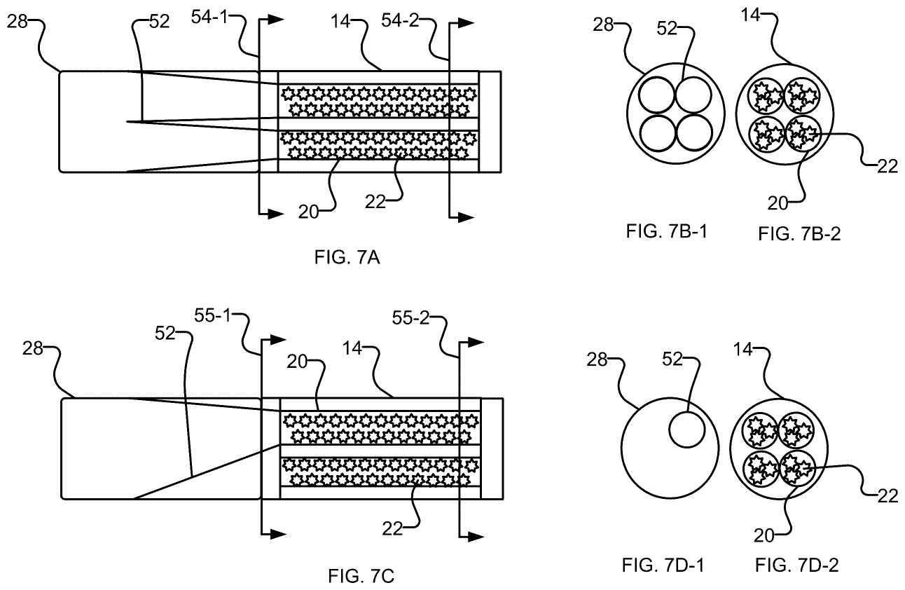

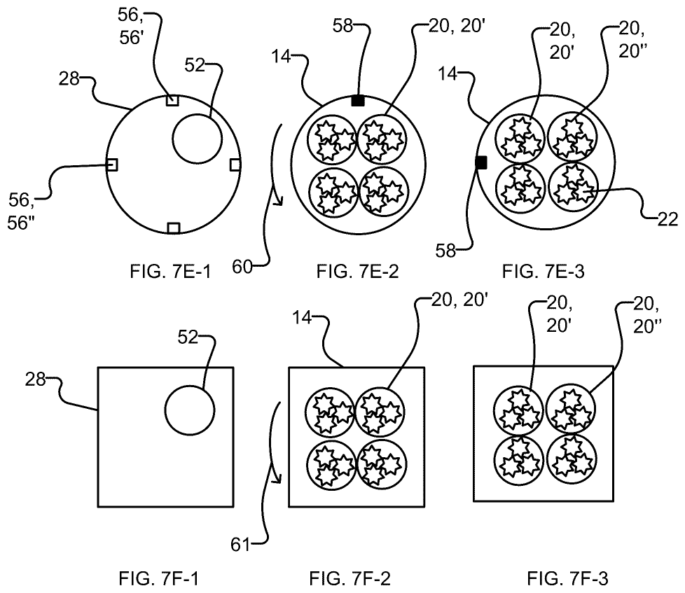

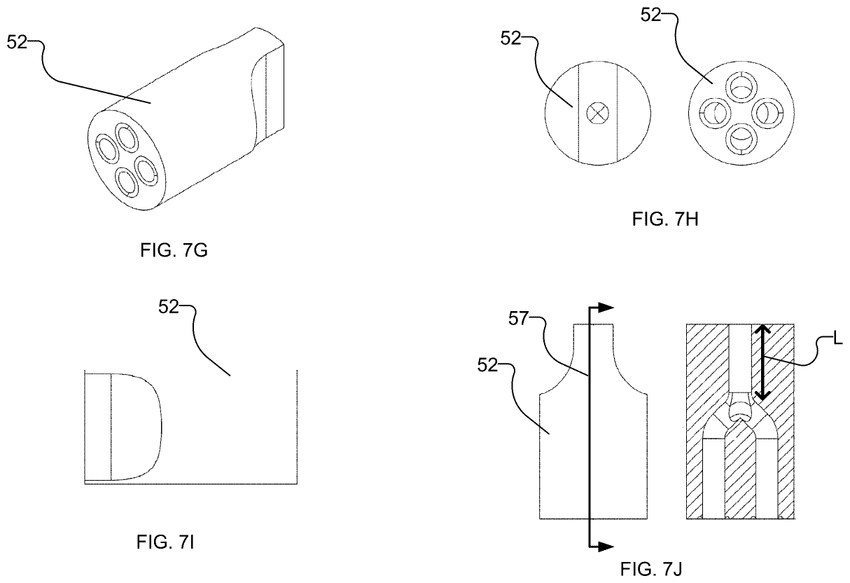

FIG. 2A depicts cross sectional views of cartridges 14 taken in a plane that is generally perpendicular to the longitudinal extension of the FIG. 1 cartridge 14 according to a number of example embodiments. As shown in FIG. 2A, there can be any number of bores 20 in cartridge 14 for accommodating any number of the same or different sample media 22. Furthermore, bores 20 may have any cross section, and need not have a uniform cross section along the length of cartridge 14. Although cartridges 14 are depicted with a round cross section in FIG. 2A, cartridge 14 could have any shaped cross section. By way of non-limiting example, cartridge 14 could have a rectangular cross section (as shown in the FIG. 7F example embodiment).

FIG. 2B is an exploded plan view of cartridge 14 according to an example embodiment. After gas sample 12 has adsorbed onto sample media 22, cartridge 14 may be transported to a piece of analytical equipment suitable for analyzing the contents of analytes from gas sample 12 that are adsorbed onto sample media 22 (for example, a mass spectrometer, a gas chromatograph and/or the like). Sometimes it is not possible or desirable to collect gas sample 12 at a location proximate to a suitable piece of analytical equipment. Consequently, in some embodiments cartridge 14 may be used to take a sample in a first location and then transported to a second location, where the sample may be analyzed. To avoid contamination with the surrounding environment, or loss of sample media 22 during transportation, in some embodiments, cartridge 14 can be detached from apparatus 10 and hermetically sealed with caps 24, and optional washer seals 25. Caps 24 may connect to cartridge 14 via threads (as shown in the depicted embodiment of FIG. 2B), or through any other fixation mechanism including (but not limited to) glue, magnets, or tape.

When transporting cartridge 14, it may be desirable to keep track of one or more parameters during transit. By way of non-limiting example, the desire to track one or more parameters during transit may be because sample media 22 may degrade under certain environmental conditions. As such, in some embodiments cartridge 14 and/or caps 24 may also comprise a temperature detector/sensor (e.g.: a non-reversible temperature sticker that indicates if a certain temperature is reached, an embedded thermocouple and/or any other suitable temperature sensor), a shock detector (e.g.: a non-reversible sticker that indicates cartridge 14 has been exposed to a certain level of acceleration, and/or any other suitable accelerometer or shock detector), and/or a geolocator module (e.g. a GPS sensor and/or the like). These are non-limiting examples of detectors and sensors which may be provided as part of cartridge 14 and/or caps 24, and there may be other detectors and/or sensors that could be used in cartridge 14 and/or caps 24. In some embodiments, there may be a barcode, an RFID tag and/or some other suitable form of identifier on cartridge 14 and/or caps 24 to facilitate inventory management with multiple cartridges 14.

FIG. 2C is a cross sectional view of cap 24 according to an example embodiment. Optional washer seal 25 rests inside of cap 24 so that when washer seal 25 is affixed to cartridge 14, washer seal 25 is in direct contact with sample media 22. In some embodiments, washer seal 25 is made of an inert material to avoid contaminating sample media 22. An example of an appropriate inert material is polytetrafluoroethylene (PTFE).

FIG. 2D is a schematic plan view of washer seal 25 according to an example embodiment. In the depicted embodiment of FIG. 2D, washer seal 25 is intended for use with a cartridge with four bores 20.

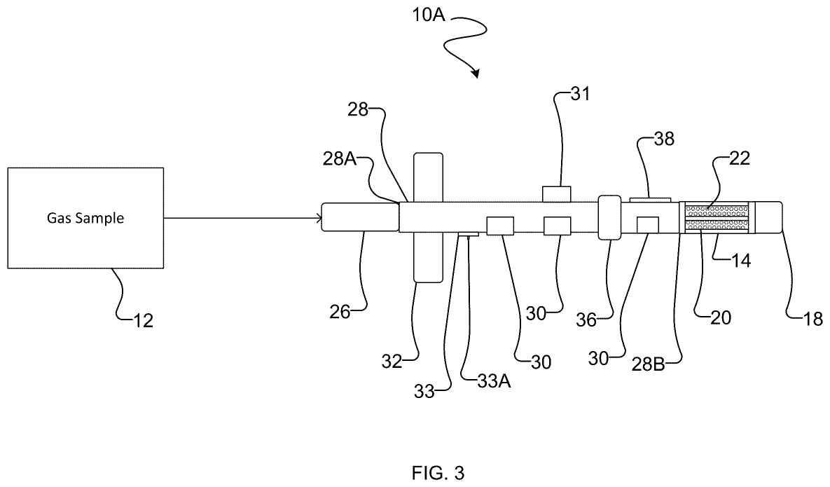

FIG. 3 is a gas sampling apparatus 10A according to an example embodiment. Unless the context dictates otherwise, those elements of apparatus 10A that are identified by references also used to identify elements of apparatus 10 (in FIG. 1) have the same or similar features and/or functions as described with respect to apparatus 10.

Gas sampling apparatus 10A comprises an interface 26 and a conduit 28. Conduit 28 forms a fluid passage. In the illustrated embodiment of FIG. 3, interface 26 is fluidly connected to conduit 28 at a first end 28A of conduit 28 and conduit 28 is fluidly connected to bores 20 of cartridge 14 at a second end 28B of conduit 28.

In some embodiments, cartridge 14 is detachably affixed to second end 28B of conduit 28 by way of a threaded coupling, a magnet, and/or the like.

At least one purpose of interface 26 is to ensure an airtight seal with gas sample 12. As mentioned above, in some embodiments gas sample 12 may be a human's exhaled breath. As such, interface 26 may comprise a mouthpiece suitable for a human to exhale into. In some embodiments, interface 26 is detachable from conduit 28 and can be replaced with a new interface 26. This is particularly advantageous for avoiding cross-contamination between users of apparatus 10A.

Interface 26 could also comprise (but is not limited to):
A mask suitable for capturing exhaled breath from both a human's nose and mouth;
A disposable straw or tube for capturing exhaled breath from a human's mouth;
A mask suitable for capturing exhaled breath from any type of animal with a respiratory system; or.
A connector that connects to an intubated organism's tracheal tube.

Conduit 28 of the illustrated FIG. 3 embodiment comprises at least one sensor 30. One purpose of sensor 30 is to measure one or more characteristics of gas sample 12 as it flows through conduit 28. Sensor 30 may generally comprise any sensor that is capable of measuring one or more characteristics of gas sample 12. Examples of sensor 30 include (but are not limited to): a sensor for sensing content(s) of gas sample 12, such as a carbon dioxide sensor, a nitrogen monoxide sensor, a sulfur oxide sensor, a volatile organic compound sensor (such as a sensor for alkanes, ketones, or aldehydes), a hydrogen sensor, and/or a sensor for other content(s) of gas sample 12; physical sensors for detecting physical characteristic of gas sample 12, such as a flow sensor, a pressure sensor, a temperature sensor, a moisture sensor, and/or the like; and/or any other desired sensor.

In some embodiments, apparatus 10A comprises a filter 32, for example a Flo-Guard™ Breathing Filter. One purpose of filter 32 is to filter out moisture from gas sample 12. Filtering moisture from gas sample 12 may be particularly important if gas sample 12 is exhaled human breath, as exhaled human breath is moist. Moisture can contaminate the inner surfaces of conduit 28 rendering them unsanitary and/or contributing to contamination of gas sample 12. Another purpose of filter 32 is to filter out viruses and bacteria from gas sample 12, which could also reduce the effectiveness of the adsorption of analyte in gas sample 12 onto sample media 22.

Deposition on filter 32 from gas sample 12 may contain analyte of interest. In some embodiments, filter 32 is removable (e.g. from apparatus 10A) so that filter 32 can be taken to an appropriate piece of testing equipment for analysis (for example, a gas chromatograph, a mass spectrometer, or an apparatus to facilitate polymerase chain reaction (PCR) testing such as a thermal cycler).

In some embodiments, conduit 28 is internally heated by an internal heating element (not expressly enumerated). One purpose of internal heating of conduit 28 is to reduce condensation in apparatus 10A when gas sample 12 is moist (for example, when gas sample 12 is human breath). Reducing condensation is also advantageous, as it reduces the amount of gas sample 12 that forms a liquid in conduit 28, thereby maintaining as much as possible of gas sample 12 in the gaseous phase for adsorption in cartridge 14.

In some embodiments, apparatus 10A comprises a fan 31 that is oriented to exhaust out of conduit 28. Fan 31 draws clean air into apparatus 10A to facilitate the removal of condensation in apparatus 10A, as well as to facilitate the removal of any remnants of gas sample 12 from conduit 28 prior to carrying out a subsequent test. In some embodiments, fan 31 is run continuously (including during the collection of gas sample 12) to reduce condensation inside conduit 28.

In some embodiments, apparatus 10A comprises a moisture trap 33. Moisture trap 33 is located near interface 26 and collects condensation accumulated in conduit 28 as a result of moisture in gas sample 12. Moisture trap 33 may be located at a lowermost region of apparatus 10A to facilitate collection of condensate in apparatus 10A. Moisture trap 33 may also comprise a release valve 33A that can release accumulated moisture collected in moisture trap 33. In some embodiments, filter 32 is positioned within apparatus 10A at a location upstream of moisture trap 33 (i.e. gas sample 12 flows through filter 32 before flowing past moisture trap 33). In some embodiments, moisture trap 33 is upstream of sensors 30.

Although the depicted embodiment in FIG. 3 shows conduit 28 as being longer than cartridge 14, in some embodiments conduit 28 may be shorter than cartridge 14.

In some embodiments, apparatus 10A has a display 38. Display 38 can display information collected from sensors 30. Display 38 can additionally or alternatively show a tutorial for the operation of apparatus 10A. Display 38 may additionally or alternatively display identification information which identifies the subject, time, location, conditions and/or the like of sample collection. In some embodiments there may be buttons (not pictured in FIG. 3) that can control the data displayed on display 38.

In some embodiments, apparatus 10A is battery powered.

It may be desirable to allow only a portion of gas sample 12 to flow through sample media 22. By way of example, it may be desirable to analyze only a fraction of a human's exhaled breath. The alveolar fraction of exhaled breath, which corresponds to the part of an exhaled breath with a high CO₂ concentration, contains exhaled breath that originates from the lower region of the lungs. This portion of exhaled breath can be desirable to capture, as it has a high concentration of analytes of interest.

To capture a portion of gas sample 12, conduit 28 may have a valve 36 capable of diverting gas sample 12 outside of conduit 28.

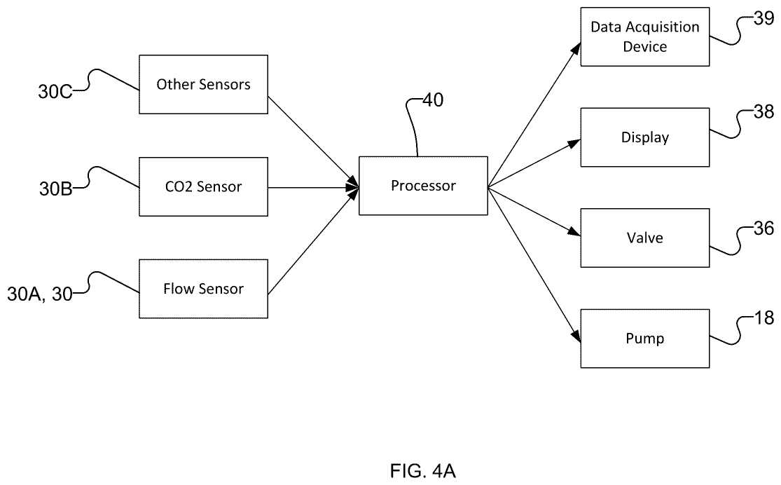

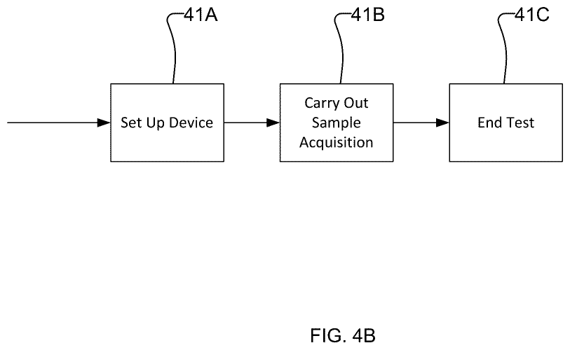

FIG. 4A shows a flowchart of inputs and outputs to a processor 40 according to an example embodiment. Processor 40 is configured to receive and process data from sensors 30 (e.g. flow sensor 30A, CO₂ sensor 30B and/or other sensors 30C in the illustrated FIG. 4A embodiment, referred to collectively as sensors 30) that have recorded one or more parameters from gas sample 12. In some embodiments processor 40 can then export data to a data acquisition device 39, such as a suitably configured computer or mobile phone executing a suitable application. In some embodiments, processor 40 can display the information obtained from sensors 30 on display 38. In some embodiments, processor 40 can use the information obtained from sensors 30 to modulate or otherwise control pressure source 18 (shown as a pump in FIG. 4A). In some embodiments, sensors 30 may include a temperature sensor, and processor 40 may control the internal heating element in conduit 28 to regulate the temperature inside conduit 28.

In some embodiments, processor 40 can obtain data from sensors 30 and use the information from sensors 30 to determine whether to actuate valve 36 open or closed to allow gas sample 12 to flow through sample media 22 in cartridge 14. In some embodiments, processor 40 can additionally or alternatively actuate pressure source 18 when valve 36 is open allow gas sample 12 to flow through sample media 22 in cartridge 14.

By way of example, if gas sample 12 is a human's exhaled breath, and it is desirable to predominantly capture the alveolar fraction of the breath, then sensors 30 could monitor the composition of the exhaled breath as the human breathes into apparatus 10A. In this example, sensor 30 could comprise a CO₂ sensor 30B, as one way of determining the alveolar fraction of exhaled breath is when there is a high (e.g. above a configurable threshold) concentration of CO₂. In some embodiments, sensor 30 could comprise a sensor for a different compound, and the concentration of the different compound could be used to determine the alveolar fraction of the exhaled breath or some other fraction having some other characteristic (e.g. a threshold characteristic) of gas sample 12.

In some embodiments, sensors 30 could measure a rate of change of a compound (for example, a rate of change of CO₂ concentration) to determine the onset of alveolar breath (e.g. identifying the alveolar fraction by identifying when the rate of change of CO₂ concentration is above or below a threshold). In some embodiments, the concentration of a compound and/or the rate of change of a compound (e.g. CO2) could be subjected to some moving average window prior to comparing it to a threshold, or be compared to a threshold for a period of time before making a decision as to whether the concentration or rate of change has crossed the threshold to minimize multiple threshold crossings which may be caused by noise and/or spurious measurements.

An example of using a gas sampling apparatus (e.g. gas sampling apparatus 10A or any of the other embodiments described herein) to gate breath sampling to sample alveolar fraction is now described, it being understood that similar techniques could be used to capture other desired fractions of other desired gas samples 12. When sensors 30 send a signal to processor 40 indicating that the exhaled breath is non-alveolar (e.g. because the CO₂ content is below a desired threshold or because of some other sensed indicia), processor 40 will send a corresponding signal to valve 36 indicating that valve 36 should open to divert the exhaled breath out of conduit 28 so that the non-alveolar portion of the exhaled breath does not flow through sample media 22. When sensors 30 send a signal to processor 40 indicating that the exhaled breath is in the alveolar stage (e.g. because the CO₂ content is below a desired threshold or because of some additional or alternative sensed indicia), processor 40 will send a corresponding signal to valve 36 indicating that valve 36 should close to allow the alveolar portion of the exhaled breath to flow through sample media 22. It will be appreciated that the operation of valve 36 could be reversed (e.g. to pass the gas sample 12 to sample media 22 when open and to divert the gas sample 12 away from sample media 22 when closed).

The alveolar (e.g. CO₂ concentration) threshold among people may vary. For example, some people may exhale alveolar breath with a CO₂ concentration lower than others. Consequently, in some embodiments the processor can be calibrated by having a test subject exhale into apparatus 10A. A user and/or operator of apparatus 10A can then view data collected by sensors 30 that has been exported to data acquisition device 39, and determine what the alveolar threshold is for the test subject. In some applications, the alveolar threshold for a particular subject may be ascertained or estimated by some other suitable technique. The user and/or operator of apparatus 10A can then modify the threshold that processor 40 uses to determine when the exhaled breath is alveolar. By way of example, the threshold could be a particular concentration of CO₂ measured by sensors 30, or a particular rate of change in CO2 concentration. The alveolar threshold can be modified via buttons on display 38, or by way of a computer or a mobile application connected to processor 40. It will be appreciated that a plurality of threshold parameters could be similarly calibrated for use in gating the fraction(s) of gas sample 12 that are provided to sample media 22.

In some embodiments, sensors 30 may also be used to keep track of sample metrics regarding gas sample 12. In particular, if gas sample 12 is exhaled human breath, then sensors 30 could track sample metrics which may include, by way of non-limiting example, the total exhalation volume, the number of breaths exhaled into apparatus 10A, the moisture content of the breaths and/or the like. Data collected from sensors 30 may be exported to data acquisition device 39 to facilitate monitoring and/or other analysis of sample metrics.

Data from sensors 30 is advantageous to collect as it supplements the analysis of sample media 22 when analyzed in analytical equipment (such as in a gas chromatograph or a mass spectrometer). Concurrent measurement (with sensors 30) and capturing (with cartridge 14) of analytes in gas sample 12 provides additional information to the user of apparatus 10A to facilitate their analysis of gas sample 12.

FIG. 4B is a flowchart of a method for operation of apparatus 10A according to an example embodiment.

Step 41A comprises setting up apparatus 10A. In a computer or mobile phone application connected to apparatus 10A, an operator may input test information, such as the operator name, study identification number, time of the sample collection, location of the sample collection and/or the like. Inputting this information may be facilitated by a barcode, an RFID tag and/or the like that is affixed to cartridge 14 and/or a test subject's information sheet that allows the computer to collect information from a database and populate data in the computer or phone application.

Step 41A can also comprise the selection of test parameters. By way of example, an operator can select whether they wish to sample a test subject's entire exhalation, or just the alveolar fraction of their breath. The operator can also select whether they wish to collect a particular volume of exhaled breath, or a particular number of exhaled breaths. One advantage of the configuration of cartridge 14 is that it is agnostic to testing schemes; an operator is not limited to testing a particular volume of gas sample 12 as they would be in devices that make use of a gas sampling bag or the like.

Step 41B comprises carrying out the sample acquisition. Display 38 may show information to the test subject instructing them on how to breathe into apparatus 10A, and instructing them when the sample acquisition is complete. During step 41B, gas sample 12 (exhaled breath, in this case) flows through apparatus 10A and valve 36 may open or close to divert a portion of the exhaled breath towards cartridge 14 (if, for example, only the alveolar fraction of the exhaled breath is desired to be captured). Step 41B may also comprise notifying the operator when the sample acquisition is complete (for example, if the operator indicated they wished to capture N breaths (where Nis an integer greater than 0), apparatus 10A could notify the operator when the test subject has completed exhaling their N^{th} breath).

Step 41C comprises exporting the data collected by sensors 30 in apparatus 10A to a data acquisition device (shown as element 39 in FIG. 4A). The data acquisition device may comprise a computer or a mobile phone executing a suitable application. In step 41C, an operator of apparatus 10A may also detach cartridge 14 from apparatus 10A and seal cartridge 14 with caps 24.

When sampling gas sample 12, it may be desirable to quickly compare data collected from sensors 30 and the data obtained from testing sample media 22 in an appropriate piece of testing equipment (such as a gas chromatograph or a mass spectrometer). Consequently, in some embodiments step 41C may further comprise associating data obtained from sensors 30 in apparatus 10A with a sample tag or element, which in some embodiments may comprise a barcode, an RFID tag and/or the like affixed to cartridge 14. Upon testing sample media 22 in an appropriate piece of testing equipment as described herein, data from the testing equipment may also be associated with the sample tag or element. Associating information obtained from sensors 30 with information obtained from testing equipment through the use of a sample tag or element facilitates analysis of gas sample 12.

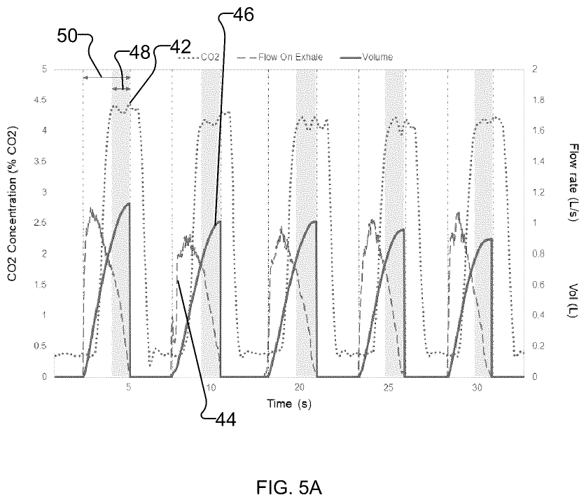

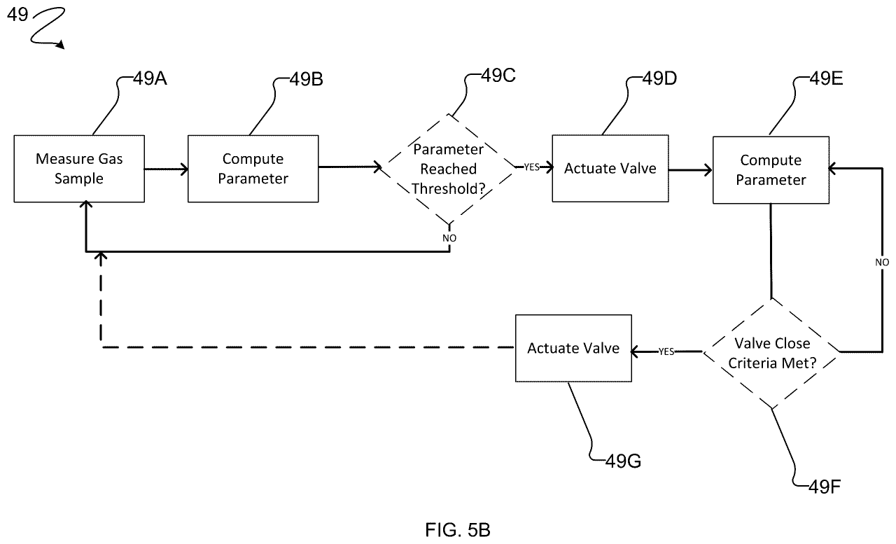

FIG. 5A is a graph of volume, flow rate, and CO₂ concentration as a function of time according to an example embodiment. In FIG. 5A, gas sample 12 is exhaled human breath. The data shown in FIG. 5A was obtained from apparatus 10A using sensors 30, and five exhalations were exhaled into apparatus 10A. Curve 42 represents the measured CO₂ volume fraction of gas sample 12 as a function of time (e.g. as measured or otherwise detected by sensors 30). Curve 44 represents measured flow rate (in L/s) of gas sample 12 as a function of time (e.g. as measured or otherwise detected by sensors 30). Curve 46 represents a measured volume of gas sample 12 collected for each exhalation (L) as a function of time (e.g. as measured or otherwise detected by sensors 30).

The time period of the first exhalation is shown as region 50 of FIG. 5A, which corresponds to an entire exhaled breath (i.e. from full lungs to the time just before starting to inhale again).

Region 48 shown in FIG. 5A represents the time period of the first exhalation corresponding to the alveolar fraction of the first exhalation. During the alveolar fraction of the exhalation (represented by region 48), the CO₂ concentration (represented by curve 42) is elevated.

FIG. 5B shows a method 49 for capturing a portion of a gas sample according to an example embodiment. Step 49A comprises taking a measurement of a gas sample, for example with sensors 30. Step 49B comprises determining a parameter based at least in part on at least the measurements from the gas sample. In some embodiments, the parameter may be a concentration of a particular compound in gas sample 12 (for example CO₂). In some embodiments, the parameter may be a rate of change of a particular parameter in gas sample 12 (for example, a rate of change of the concentration of CO₂). The rate of change may optionally be averaged with a moving average filter or a temporal threshold to remove noise and/or spurious measurements. Step 49C comprises comparing the block 49B determined parameter to a threshold. In some embodiments, the threshold may be a particular concentration of a particular compound and/or rate of change of some other parameter (for example CO₂ concentration (see curve 42 of FIG. 5A)). In some embodiments, the threshold may be a rate of change of a particular parameter (by way of example, referring to region 48 of FIG. 5A, when the alveolar fraction is reached the rate of change of CO₂ concentration decreases). The threshold may be optionally adjusted by a user or operator. If the step 49C inquiry is negative, then method 49 loops back to step 49A. If on the other hand, the step 49C inquiry is positive, then method 49 proceeds to step 49D. Step 49D comprises actuating (e.g. opening) valve 36, so that the portion of gas sample 12 to be collected is diverted to cartridge 14 (and to sample media 22).

When valve 36 is actuated to allow the portion of gas sample 12 to be collected to be diverted to cartridge 14, it may be desirable to actuate valve 36 again (e.g. to close valve 36) once the portion of gas sample 12 to be collected (e.g. in a particular exhalation) has been collected. Consequently, in some embodiments step 49E may comprise determining a second parameter after valve 36 has been actuated. In some embodiments, the parameter may be a rate of change of a compound or rate of change of some other parameter (for example CO₂ concentration (see curve 42 of FIG. 5A) or measured volume of gas sample 12 collected for each exhalation (see curve 46 of FIG. 5A)); as visible from FIG. 5A, the rate of change of CO2 (curve 42) decreases sharply at the end of the alveolar portion of the exhalation represented by region 48 as does the rate of change of volume in a particular exhalation (curve 46). In some embodiments, the parameter may be a change in time (e.g. a time interval) from when valve 36 was initially actuated (e.g. in step 49D) to divert the portion of gas sample 12 to be collected to cartridge 14. In some embodiments, the step 49E parameter may be measured flow rate. Step 49F comprises comparing the step 49E determined parameter to a valve close criteria that indicates when the portion of gas sample 12 to be collected has been collected or that the fraction of gas sample 12 to be sampled should otherwise stop. If the step 49F inquiry is positive (i.e. if the valve close criteria has been met), then method 49 proceeds to step 49G which comprises actuating (e.g. closing) valve 36 again so that the gas sample is diverted outside of conduit 28. If the step 49F inquiry is negative (i.e. if the valve close criteria has not been met), then valve 36 does not close and gas sample 12 is permitted to flow through cartridge 14). The method 49 procedure up until step 49G permits gating the sampling of gas sample 12 based on particular criteria (e.g. measured and/or determined parameters). It may be desirable to use such gating to capture gas from particular portions of an exhalation. Method 49 may optionally loop from step 49G back to step 49A to use valve 36 to gate another exhalation.

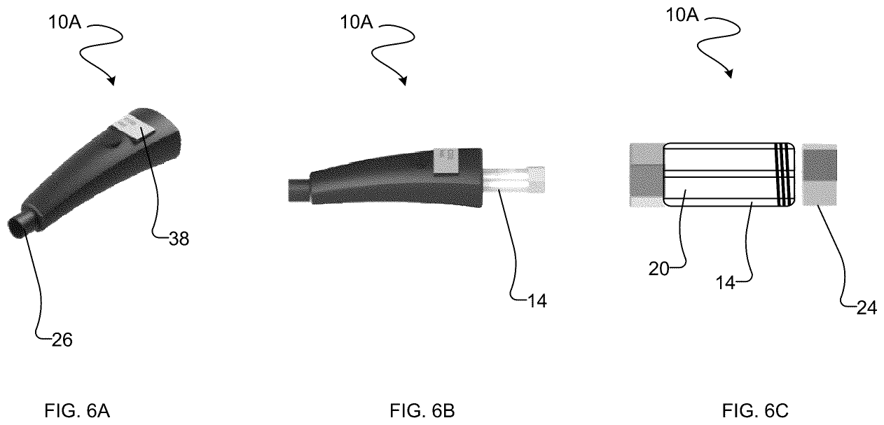

FIG. 6A, FIG. 6B, and FIG. 6C show a prototype gas sampling apparatus 10A according to an example embodiment. FIG. 6A is a perspective view of apparatus 10A. FIG. 6B is a side view of apparatus 10A. FIG. 6C is a side view of cartridge 14.

FIGS. 7A, 7B-1 and 7B-2 show connection interfaces between conduit 28 and cartridge 14 in apparatus 10A according to an example embodiment. Baffle 52 directs the flow of gas sample 12 from conduit 28 into bores 20 of cartridge 14. FIG. 7B-1 is a cross sectional view of the FIG. 7A apparatus 10A in the plane defined by line 54-1 and FIG. 7B-2 is a cross-sectional view of the FIG. 7A apparatus 10A in the plane defined by line 54-2. In FIGS. 7A and 7B, all bores 20 of cartridge 14 are exposed to gas sample 12 at the same time.

In some embodiments, baffle 52 is configured to distribute gas sample 12 uniformly through cartridge and/or the bore(s) 20 of cartridge 14. Uniform distribution of gas sample 12 is advantageous as it ensures samples collected in cartridge 14 are collected under the same conditions.

It may be advantageous to test a gas sample 12 using the same or different types of sample media 22 in quick succession. It may also be advantageous to serially test different gas samples 12 in quick succession.

FIGS. 7C, 7D-1 and 7D-2 show connection interfaces between conduit 28 and cartridge 14 in apparatus 10A according to an example embodiment. FIG. 7D-1 is a cross sectional view of the FIG. 7C apparatus 10A in the plane defined by line 55-1 and FIG. 7D-2 is a cross-sectional view of the FIG. 7C apparatus 10A in the plane defined by line 55-2.

Baffle 52 of the FIG. 7C and FIG. 7D embodiment is configured (e.g. shaped) to direct the flow of gas sample 12 from conduit 28 through one bore 20 of cartridge 14. Using the connection interfaces shown in FIGS. 7C and 7D, a user can test one gas sample 12 at a time by exposing gas sample 12 to only one of bores 20 in cartridge 14. After exposing gas sample 12 to one of bores 20, the user can detach, rotate, or move cartridge 14 relative to conduit 28 so that a different bore 20 lines up with baffle 52.

Although in the embodiment of FIG. 7C and FIG. 7D, baffle 52 is shaped to direct flow into one bore 20 of cartridge 14 at a time, in some embodiments baffle 52 could be configured (e.g. shaped) to direct flow into any number of bores 20 of cartridge 14 at a time.

It is advantageous to be able to precisely align baffle 52 with the one or more bores 20 of cartridge 14. In some embodiments, a key mechanism and/or the like may be used to align baffle 52 with one or more bores 20 of cartridge 14.

FIGS. 7E-1, 7E-2 and 7E-3 (collectively, FIG. 7E) schematically depict a key mechanism for mating conduit 28 with cartridge 14 according to an example embodiment. Receptacles 56 are shaped to accept (e.g. to be complementary to the shape of) a corresponding protrusion 58 located on cartridge 14. Protrusion 58 may be aligned such that when protrusion 58 is received in a corresponding one of receptacles 56, baffle 52 lines up with a corresponding one (or more) of bores 20 in cartridge 14. For example, when cartridge has the alignment shown in FIG. 7E-2, protrusion 58 aligns with receptable 56′, so that baffle 52 aligns with bore 20′ in cartridge 14. When protrusion 58 is received in receptacle 56, cartridge 14 may be retained in position (e.g. by a suitable closure or locking mechanism (not shown).

After passing gas sample 12 through bore 20′, cartridge 14 can be rotated in the direction represented by arrow 60 (to the configuration shown in FIG. 7E-3), such that protrusion 58 is aligned with and received in receptacle 56″. With protrusion 58 received in receptacle 56″, baffle 52 is lined up with bore 20″.

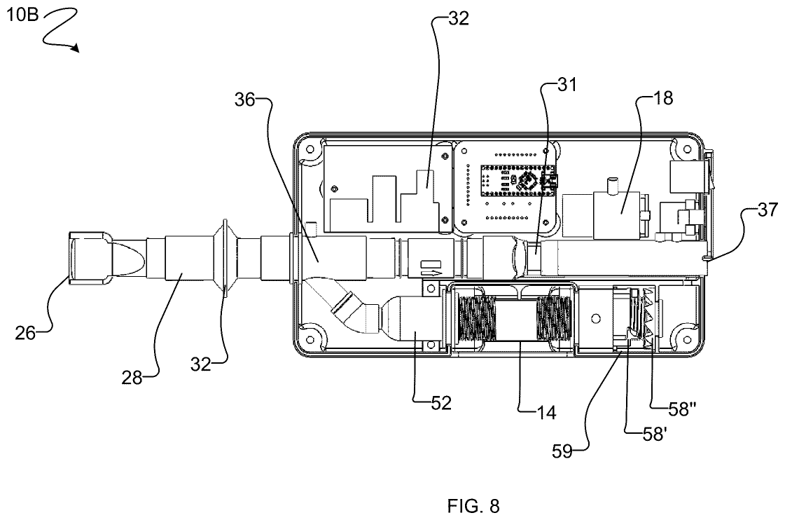

Although FIG. 7E shows a protrusion and receptacle key mechanism, other additional and/or alternative types of alignment mechanism(s) may be used to align baffle 52 with bores 20. By way of non-limiting example, a ball and socket, a ratchet and pawl, a piece of spring steel, or a bistable cam mechanism with a spring-biased step member 58′ that indexes along an intermittent stop 58″ (as shown in FIG. 8) may be used. Some such alignment mechanisms may comprise detent mechanisms which bias their respective components into engagement with one another.

It will be appreciated that there are other mechanisms for serially testing different samples using apparatus 10A, including (but not limited to) an adjustable baffle that can be configured to direct flow into different bores 20.

FIGS. 7F-1, 7F-2 and 7F-3 (collectively, FIG. 7F) schematically depict an interface between conduit 28 and cartridge 14 according to an example embodiment. Those elements in FIG. 7F that are identified by references also used to identify elements in FIG. 7E have the same or similar functions as described with respect FIG. 7E. In FIG. 7F, conduit 28 and cartridge 14 have polygonal (e.g. rectangular) cross sections that can be used to align baffle 52 with individual bores 20. After passing gas sample 12 through bore 20′ (in the configuration of FIG. 7F-2), cartridge 14 can then be rotated in the direction represented by arrow 61 such that bore 20″ lines up with baffle 52 (in the configuration of FIG. 7F-3). Rather than using a key mechanism as shown in FIG. 7E, in FIG. 7F the polygonal cross sections of conduit 28 and cartridge 14 are used to align baffle 52 with individual bores 20. Though conduit 28 and cartridge 14 are shown with rectangular cross sections in FIG. 7F, conduit 28 and cartridge 14 could have the cross section of any polygon.

FIG. 7G is a perspective view of a baffle 52 according to an example embodiment. FIG. 7H is a front view and a rear view of the baffle of FIG. 7G. FIG. 7I is a side view of the baffle of FIG. 7G. FIG. 7J is a cross sectional view of the baffle of FIG. 7G in the plane defined by line 57.

When diverting gas sample 12 from conduit 28 to bores 20 of cartridge 14, it may be advantageous to maintain laminar flow. Laminar flow ensures that gas sample 12 separates evenly to the one or more bores 20 of cartridge 14. Consequently, in some embodiments the dimensions of baffle 52 can be chosen to promote laminar flow within baffle 52. By way of example, this could be achieved by selecting interior dimensions of baffle 52 with a small diameter and a long entrance length represented by arrow L in FIG. 7J.

FIG. 8 is a partially sectioned view of a sampling apparatus 10B according to an example embodiment. Unless the context dictates otherwise, those elements of apparatus 10B that are identified by references also used to identify elements of apparatus 10 (in FIG. 1) and 10A (in FIG. 3) have the same or similar features and/or functions as described with respect to apparatus 10 and 10A.

Sampling apparatus 10B differs from sampling apparatus 10A in that sampling apparatus 10B comprises an exhaust port 37. Valve 36 may comprise a three way valve that is configured to divert gas sample 12 towards cartridge 14, or out of exhaust port 37. Valve 36 can actuate based on the measurements obtained by sensors 30, as described elsewhere herein.

In sampling apparatus 10B, pressure source 18 (shown as a pump in FIG. 8) is fluidly connected to cartridge 14 and is configured to draw gas sample 12 through cartridge 14. Apparatus 10B comprises a bistable cam mechanism 59 with a spring-biased step member 58′ that indexes along an intermittent stop 58″, analogous in function to a mechanism in a retractable pen. Spring-biased step member 58′ in cam mechanism 59 retains cartridge 14 in position while testing gas sample 12. Cam mechanism 59 also retains cartridge 14 in a position relative to conduit 28, such that one or more of bores 20 (not visible in FIG. 8) are aligned with baffle 52.

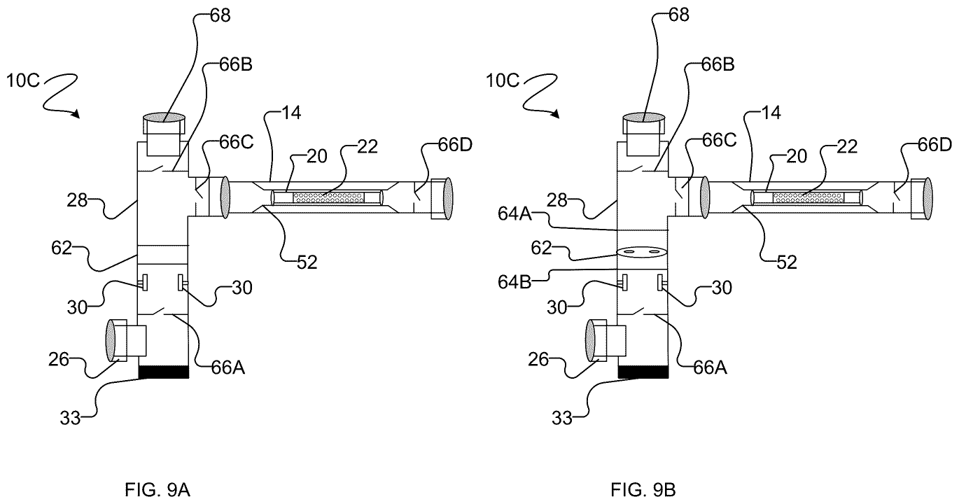

FIG. 9A and FIG. 9B depict sectional views of a gas sampling apparatus 10C according to an example embodiment. Unless the context dictates otherwise, those elements of apparatus 10C that are identified by references also used to identify elements of apparatus 10 (in FIG. 1), 10A (in FIG. 3), and 10B (in FIG. 8) have the same or similar features and/or functions as described with respect to apparatus 10, 10A, and 10B.

One way valves 66A, 66B, 66C, and 66D of apparatus 10C prevent ambient air from entering apparatus 10C. One way valves 66A, 66B, 66C, and 66D also prevent a user of apparatus 10C breathing in air from inside apparatus 10C, which may be unsanitary.

Apparatus 10C is advantageous for capturing samples of exhaled human breath. As mentioned elsewhere herein, it may be desirable to maintain a particular flow rate of gas sample 12 through sample media 22 to facilitate adsorption of analyte onto sample media 22. As a human exhales into apparatus 10C at interface 26, sensors 30 measure the flow rate of exhalation. If the flow rate of gas sample 12 is within a range that facilitates adsorption of analyte onto sample media 22, indicator 62 will signal to provide feedback. In some embodiments, indicator 62 comprises an LED that illuminates when the flow rate of gas sample 12 is within a range that facilitates adsorption of analyte onto sample media 22, as shown in FIG. 9A. In some embodiments, indicator 62 comprises a float that is kept between a first mark 64A and second mark 64B that indicates that a desired flow rate has been achieved as shown in FIG. 9B. The float is analogous in function to a rotameter. Ventilation port 68 purges excess gas sample 12, which may be desirable if the flow rate of gas sample 12 is too high for adequate adsorption.

One advantage of the configuration of apparatus 10C is that indicator 62 is directly in front of the eyes of the individual exhaling into the apparatus, facilitating visual feedback of their exhalation flow rate.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

While processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Where a component is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

The invention has a number of non-limiting aspects. Non-limiting aspects of the invention comprise:

1. An apparatus for collecting analyte from a gas sample, the apparatus comprising:
a cartridge fluidly connectable to the gas sample, the cartridge shaped to define one or more bores configurable to be in fluid communication with the gas sample; and
sample media comprising sorbent material insertable into the one or more bores;
wherein a pressure gradient draws at least a portion of the gas sample into the cartridge and into contact with the sample media.

2. The apparatus according to aspect 1 (or any other aspect herein) wherein the gas sample comprises exhaled human breath.

3. The apparatus according to aspect 2 (or any other aspect herein) wherein the pressure gradient is created at least in part by an exhalation pressure associated with the human breath.

4. The apparatus according to aspect 1 (or any other aspect herein) wherein the gas sample comprises one or more of: ambient air, outgas from a biological sample, outgas from soil, outgas from waste, or outgas from plants.

5. The apparatus according to any one of aspects 1 to 4 (or any other aspect herein) comprising a pump for creating, at least in part, the pressure gradient.

6. The apparatus according to aspect 1 (or any other aspect herein) wherein the pressure gradient is created, at least in part, by a pressure associated with the gas sample.

7. The apparatus according to aspect 5 (or any other aspect herein) wherein the pump acts directly on the gas sample or on the at least a portion of the gas sample.

8. The apparatus according to any one of aspects 1 and 6 (or any other aspect herein) wherein the gas sample is contained in an inflated gas sampling bag.

9. The apparatus according to any one of aspects 1 to 8 (or any other aspect herein) wherein the cartridge is hermetically sealable with a first cap at a first end of the cartridge, and a second cap at a second end of the cartridge opposite to the first end of the cartridge.

10. The apparatus according to aspect 9 (or any other aspect herein) wherein the first cap and the second cap comprise a first washer seal and a second washer seal.

11. The apparatus according to aspect 10 (or any other aspect herein) wherein the first washer seal and second washer seal are fabricated from an inert material.

12. The apparatus according to any one or aspects 1 to 11 (or any other aspect herein) comprising at least one detector coupled to the cartridge, the at least one detector comprising one or more of: a temperature detector, a shock detector, and a geolocator module.

13. The apparatus according to any one of aspects 1 to 12 (or any other aspect herein) wherein the sample media comprises one or more of: a thermal desorption tube, GERSTEL Twister®, Solid-Phase Microextraction fibers, Thin-Film Solid-Phase Microextraction (TF-SPME), polydimethylsiloxane (PDMS) stir bars, Markes quartz wool, Markes glass wool, and HiSorb™ probes.

14. The apparatus according to any one of aspects 1 to 13 (or any other aspect herein) comprising an identifier on the cartridge, the identifier comprising one or more of: an RFID tag, and a barcode.

15. The apparatus according to any one of aspects 1 to 14 (or any other aspect herein) comprising:
a conduit with a first end fluidly connectable to the gas sample and a second end opposite to the first end, the second end connectable to the cartridge to provide fluid communication between the gas sample and the cartridge through the conduit, the conduit further comprising:
   one or more sensors capable of measuring or otherwise detecting one or more parameters of the gas sample within the conduit.

16. The apparatus according to aspect 15 (or any other aspect herein) wherein the second end of the conduit is detachably connectable to the cartridge.

17. The apparatus according to any one of aspects 15 to 16 (or any other aspect herein) wherein the second end of the conduit is threadably connectable to the cartridge.

18. The apparatus according to any one of aspects 15 to 17 (or any other aspect herein) comprising an interface at the first end for facilitating the connection between the first end and the gas sample, wherein the interface comprises one of: a mouthpiece, a straw, a mask for a human, a mask for an animal, and a connector for a tracheal tube.

19. The apparatus according to aspect 18 (or any other aspect herein) wherein the interface is detachably connectable to one or more of: the gas sample and the conduit.

20. The apparatus according to any one of aspects 15 to 19 (or any other aspect herein) wherein the conduit comprises a filter located in a fluid passage of the conduit between the gas sample and the cartridge.

21. The apparatus according to aspect 20 (or any other aspect herein) wherein the filter is in a position in the fluid passage of the conduit closer to the first end of the conduit than a moisture trap.

22. The apparatus according to aspect 20 or aspect 21 (or any other aspect herein) wherein the filter is removable from the apparatus.

23. The apparatus according to any one of aspects 15 to 22 (or any other aspect herein) comprising a fan in fluid communication with a fluid passage of the conduit between the gas sample and the cartridge.

24. The apparatus according to any one of aspects 15 to 23 (or any other aspect herein) comprising a heating element for providing heat in a fluid passage of the conduit between the gas sample and the cartridge.

25. The apparatus according to any one of aspects 15 to 24 (or any other aspect herein) comprising a moisture trap located to remove moisture from a fluid passage of the conduit between the gas sample and the cartridge.

26. The apparatus according to aspect 25 (or any other aspect herein) wherein the moisture trap is located in a position in the fluid passage of the conduit closer to the first end of the conduit than the one or more sensors.

27. The apparatus according to according to any one of aspects 15 to 26 (or any other aspect herein) comprising a display for outputting indicia corresponding to one or more parameters measured with the one or more sensors.

28. The apparatus according to any one of aspects 15 to 27 (or any other aspect herein) comprising a valve connected to the conduit convertible between a first configuration where gas is permitted to flow between the gas sample and the cartridge through the conduit and a second configuration where the gas sample is diverted before reaching the cartridge.

29. The apparatus according to aspect 28 (or any other aspect herein) comprising a processor, the processor connected to receive one or more signals from the one or more sensors, configured to determine a portion of the gas sample to be sampled based at least in part on the one or more signals, and connected to actuate the valve between the first configuration and the second configuration based at least in part on the determined portion of the gas.

30. The apparatus of aspect 29 (or any other aspect herein) wherein the processor is further configured to actuate a pump to impose the pressure gradient when the valve is in the first configuration.

31. The apparatus according to aspect 29 or 30 (or any other aspect herein) wherein the portion of the gas sample to be sampled is an alveolar portion of a human's exhaled breath.

32. The apparatus according to aspect 31 (or any other aspect herein) wherein the processor is configured to determine one or more thresholds associated with determining the alveolar portion in a calibration operation of the apparatus based at least in part on the human's exhaled breath.

33. The apparatus according to aspect 32 (or any other aspect herein) wherein the one or more thresholds is a concentration of CO2.

34. The apparatus according to aspect 32 (or any other aspect herein) wherein the one or more thresholds is a rate of change of concentration of CO2.

35. The apparatus according to any one of aspects 15 to 34 (or any other aspect herein) comprising a baffle located in a fluid passage of the conduit between the gas sample and the cartridge, the baffle shaped to direct fluid to a subset of the one or more of the bores in the cartridge.

36. The apparatus according to aspect 36 wherein the baffle is configurable between a first position that directs fluid to a first subset of the one or more bores in the cartridge, and a second position that directs fluid to a second subset of the one or more bores.

37. The apparatus according to aspect 35 (or any other aspect herein) further comprising a key mechanism configured (e.g. shaped and/or located) to orient the cartridge in a first orientation relative to the conduit, so that the baffle is located to direct gas from the gas sample to a first subset of the one or more of the bores in the cartridge.

38. The apparatus according to aspect 37 (or any other aspect herein) wherein the key mechanism is further configured (e.g. shaped and/or located) to orient the cartridge in a second orientation relative to the conduit, so that the baffle is located to direct gas from the gas sample to a second subset of the one or more bores in the cartridge, the second subset of bores different from the first subset of bores.

39. The apparatus according to aspect 35 (or any other aspect herein) wherein cross sections of the conduit and the cartridge are shaped to provide complementary polygonal shapes, and wherein the cross sections of the conduit and the cartridge are alignable in a first relative orientation where the baffle is located to direct gas from the gas sample to a first subset of the one or more bores in the cartridge.

40. The apparatus according to aspect 39 (or any other aspect herein) wherein the cross-sections of the conduit and the cartridge are alignable in a second relative orientation where the baffle is located to direct gas from the gas sample to a second subset of the one or more bores in the cartridge, the second subset of bores different from the first subset of bores.

41. The apparatus according to any one of aspects 35 to 40 (or any other aspect herein) wherein the baffle is shaped and/or dimensioned to promote laminar flow of the gas sample through a fluid passage of the conduit between the gas sample and the cartridge.

42. The apparatus according to any one of aspects 15 to 41 (or any other aspect herein) wherein the apparatus is battery powered.

43. The apparatus according to any one of aspects 15 to 42 (or any other aspect herein) wherein data from the one or more sensors is exportable to a data acquisition device.

44. The apparatus according to aspect 43 (or any other aspect herein) wherein data exported to the data acquisition device is associated with a sample tag affixed to or otherwise connected to the cartridge.

45. The apparatus according to aspect 44 (or any other aspect herein) wherein the sample tag comprises an RFID tag or a barcode.

46. The apparatus according to aspect 44 or 45 (or any other aspect herein) wherein the data obtained from analyzing the sample media in the cartridge is associated with the sample tag.

47. The apparatus according to any one of aspects 15 to 46 (or any other aspect herein) wherein one or more of the sensors comprises a flow sensor for detecting a rate of flow of the gas from the gas sample to the cartridge, and wherein the apparatus comprises an indicator that indicates whether a particular flow rate has been achieved based on an output from the flow sensor.

48. The apparatus according to aspect 47 (or any other aspect herein) wherein the indicator comprises one or more of: an LED, and a rotameter.

49. The apparatus according to any one of aspects 15 to 48 (or any other aspect herein) comprising at least one one way valve in the conduit and convertible between a first configuration where gas is permitted to flow between the gas sample and the cartridge through the conduit and a second configuration where gas from the gas sample is prevented from reaching the cartridge.

50. The apparatus according to any one of aspects 15 to 49 (or any other aspect herein) comprising a ventilation port connected to purge excess gas from the gas sample.

51. The apparatus of any one of aspects 1 to 50 (or any other aspect herein) comprising:
a conduit with a first end fluidly connectable to the gas sample and a second end opposite to the first end connectable to a three way valve;
an interface that fluidly connects the gas sample to the first end of the conduit;
an exhaust port fluidly connected to a first exit of the three way valve;
a baffle fluidly connected to a second exit of the three way valve, the baffle being shaped to direct fluid to at least one of the one or more of the bores in the cartridge; and
one or more sensors capable of measuring or otherwise detecting one or more parameters of the gas sample within the conduit.

52. The apparatus of aspect 51 (or any other aspect herein) wherein the baffle is at least one of: configurable between a first position that directs fluid to a first subset of the one or more bores in the cartridge, and a second position that directs fluid to a second subset of the one or more bores; and shaped to direct fluid to a selectable subset of the one or more of the bores in the cartridge.

53. The apparatus of any one of aspects 51 to 52 (or any other aspect herein) wherein the interface is detachably connectable to one or more of: the gas sample and the first end of the conduit.

54. The apparatus of any one of aspects 51 to 53 (or any other aspect herein) wherein the conduit comprises a filter located in a fluid passage of the conduit between the gas sample and the first ends of the conduit.

55. The apparatus of any one of aspects 51 to 54 (or any other aspect herein) comprising a processor, the processor connected to receive one or more signals from the one or more sensors, configured to determine a portion of the gas sample to be sampled based at least in part on the one or more signals and connected to actuate a three-way valve between a first configuration where gas is permitted to flow between the gas sample and the cartridge through the conduit and a second configuration where gas from the gas sample is prevented from reaching the cartridge.

56. The apparatus of aspect 55 (or any other aspect herein) wherein the portion of the gas to be sampled is an alveolar fraction of a human's exhaled breath.

57. The apparatus of any one of aspects 51 to 56 (or any other aspect herein) comprising a fan in fluid communication with the exhaust port.

58. The apparatus of any one of aspects 51 to 57 (or any other aspect herein) further comprising a detent mechanism configured to retain the cartridge in a position relative to the conduit that aligns the baffle with one or more of the one or more bores in the cartridge.

59. The apparatus of aspect 58 (or any other aspect herein) wherein the detent mechanism comprises a bistable cam mechanism with a spring biased step member that indexes along an intermittent stop.

60. The apparatus according to any one or aspects 51 to 59 (or any other aspect herein) wherein data from the one or more sensors is exportable to a data acquisition device.

61. A method of collecting analyte from a gas sample comprising:
drawing at least a portion of the gas sample through one or more bores in a cartridge with a pressure gradient wherein the bores are filled with sample media; and
adsorbing analyte in the gas sample onto the sample media.

62. The method of aspect 61 (or any other aspect herein) further comprising imposing the pressure gradient directly on the gas sample or on the at least a portion of the gas sample with a pump.

63. The method of aspect 61 (or any other aspect herein) further comprising:
attaching a first end of a conduit to the gas sample;
attaching a second end of the conduit to the cartridge so as to provide fluid communication between the gas sample and the cartridge through the conduit; and
measuring one or more parameters of the gas sample in the conduit with one or more sensors.

64. The method of aspect 63 (or any other aspect herein) further comprising
analyzing at least the one or more parameters of the gas sample within the conduit with a processor to determine a portion of the gas sample to be captured; and
actuating a valve between:
   a first configuration where gas is permitted to flow between the gas sample and the cartridge through the conduit; and
   a second configuration where gas is diverted before reaching the cartridge;
      such that the portion of the gas sample to be captured is permitted to flow between the gas sample and the cartridge through the conduit.

65. The method of aspect 64 (or any other aspect herein) further comprising analyzing the one or more parameters of the gas sample to determine when a concentration of a compound has been reached.

66. The method of aspect 64 (or any other aspect herein) further comprising analyzing the one or more parameters of the gas sample to determine when a rate of change of concentration of a compound has been reached.

67. The method of aspect 64 (or any other aspect herein) further comprising actuating the valve from the first configuration to the second configuration when the gas sample to be captured has flowed through the sample media in the cartridge.

68. The method of aspect 67 (or any other aspect herein) comprising actuating the valve from the first configuration to the second configuration when one or more of the following are fulfilled: a rate of change of a compound is reached; a time interval has passed since the valve actuated from the second configuration to the first configuration; and when a volumetric flow rate is reached.

69. The method of aspect 63 (or any other aspect herein) further comprising:
sensing a flow rate of gas in the cartridge with one or more sensors; and
indicating whether a particular flow rate has been achieved based at least on the measurement of the one or more sensors.

70. The method of aspect 69 (or any other aspect herein) wherein indicating whether the particular flow rate has been achieved comprises illuminating an LED.

71. The method of aspect 70 (or any other aspect herein) further wherein indicating whether the particular flow rate has been achieved comprises a float balanced between a first mark and a second mark.

72. The method of aspect 61 (or any other aspect herein) further comprising
attaching a first end of a conduit to the gas sample;
attaching a second end of the conduit to a three way valve;
attaching the cartridge to a first exit of the three way valve;
attaching an exhaust port to a second exit of the three way valve; and measuring one or more parameters of the gas sample within the conduit with one or more sensors.

73. The method of aspect 72 (or any other aspect herein) further comprising:
analyzing the one or more parameters of the gas sample within the conduit with a processor to determine a portion of the gas sample to be captured; and
actuating the three way valve between:
   a first configuration where gas is permitted to flow between the gas sample and the cartridge through the conduit; and
   a second configuration where gas is permitted to flow through the exhaust port;
such that the portion of the gas sample to be captured is permitted to flow between the gas sample and the cartridge through the conduit.

74. The method of aspect 73 (or any other aspect herein) further comprising analyzing the one or more parameters of the gas sample to determine when a concentration of CO₂ has been reached.

75. The method of aspect 74 (or any other aspect herein) further comprising analyzing the one or more parameters of the gas sample to determine when a rate of change of concentration of CO₂ has been reached.

76. An apparatus having any combination or sub combination of features or elements as described herein.

77. A method comprising any step, act, combination of steps and/or acts or sub combination of steps and/or acts as described herein.

78. A non-transitory machine readable medium storing instructions that when executed by a data processor cause the data processor to perform a control method for an apparatus as described herein.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. An apparatus for collecting analyte from a gas sample, the apparatus comprising:
a cartridge fluidly connectable to the gas sample, the cartridge shaped to define one or more bores configurable to be in fluid communication with the gas sample;
sample media comprising sorbent material insertable into the one or more bores;
a conduit with a first end fluidly connectable to the gas sample and a second end opposite to the first end, the second end connectable to the cartridge to provide fluid communication between the gas sample and the cartridge through the conduit, the conduit further comprising:
one or more sensors capable of measuring or otherwise detecting one or more parameters of the gas sample within the conduit;
wherein a pressure gradient draws at least a portion of the gas sample into the cartridge and into contact with the sample media; and
wherein the second end of the conduit is detachably connectable to the cartridge.

2. The apparatus according to claim 1 wherein the cartridge is hermetically sealable with a first cap at a first end of the cartridge, and a second cap at a second end of the cartridge opposite to the first end of the cartridge.

3. The apparatus according to claim 2 wherein the first cap and the second cap comprise a first washer seal and a second washer seal, wherein the first washer seal and second washer seal are fabricated from an inert material.

4. The apparatus according to claim 1 comprising at least one detector coupled to the cartridge, the at least one detector comprising one or more of: a temperature detector, a shock detector, and a geolocator module.

5. The apparatus according to claim 1 comprising an identifier on the cartridge, the identifier comprising one or more of: an RFID tag, and a barcode.

6. The apparatus according claim 1 comprising a moisture trap located to remove moisture from a fluid passage of the conduit between the gas sample and the cartridge.

7. The apparatus according to claim 6 wherein the moisture trap is located in a position in the fluid passage of the conduit closer to the first end of the conduit than the one or more sensors.

8. The apparatus according to claim 1 comprising:
a valve connected to the conduit convertible between a first configuration where gas is permitted to flow between the gas sample and the cartridge through the conduit and a second configuration where the gas sample is diverted before reaching the cartridge; and
a processor, the processor connected to receive one or more signals from the one or more sensors, configured to determine a portion of the gas sample to be sampled based at least in part on the one or more signals, and connected to actuate the valve between the first configuration and the second configuration based at least in part on the determined portion of the gas.

9. The apparatus according to claim 8 wherein the portion of the gas sample to be sampled is an alveolar portion of a human's exhaled breath.

10. The apparatus according to claim 9 wherein the processor is configured to determine one or more thresholds associated with determining the alveolar portion in a calibration operation of the apparatus based at least in part on the human's exhaled breath.

11. The apparatus according to claim 10 wherein the one or more thresholds is one or more of: (a) a concentration of CO2, or (b) a rate of change of concentration of CO2.

12. The apparatus according to claim 1 comprising a baffle located in a fluid passage of the conduit between the gas sample and the cartridge, the baffle shaped to direct fluid to a subset of the one or more of the bores in the cartridge.

13. The apparatus according to claim 12 wherein the baffle is configurable between a first position that directs fluid to a first subset of the one or more bores in the cartridge, and a second position that directs fluid to a second subset of the one or more bores.

14. The apparatus according to claim 12 further comprising a key mechanism configured to orient the cartridge in a first orientation relative to the conduit, so that the baffle is located to direct gas from the gas sample to a first subset of the one or more of the bores in the cartridge, wherein the key mechanism is further configured to orient the cartridge in a second orientation relative to the conduit, so that the baffle is located to direct gas from the gas sample to a second subset of the one or more bores in the cartridge, the second subset of bores different from the first subset of bores.

15. The apparatus according to claim 1 wherein data from the one or more sensors is exportable to a data acquisition device and is associated with a sample tag affixed to or otherwise connected to the cartridge.

16. The apparatus according to claim 15 wherein the sample tag comprises an RFID tag or a barcode.

17. The apparatus according to claim 15 wherein data obtained from analyzing the sample media in the cartridge is associated with the sample tag.

18. An apparatus for collecting analyte from a gas sample, the apparatus comprising:
a cartridge fluidly connectable to the gas sample, the cartridge shaped to define one or more bores configurable to be in fluid communication with the gas sample;
sample media comprising sorbent material insertable into the one or more bores;
wherein a pressure gradient draws at least a portion of the gas sample into the cartridge and into contact with the sample media;
a conduit with a first end fluidly connectable to the gas sample and a second end opposite to the first end connectable to a three way valve;
an interface that fluidly connects the gas sample to the first end of the conduit;
an exhaust port fluidly connected to a first exit of the three way valve;
a baffle fluidly connected to a second exit of the three way valve, the baffle being shaped to direct fluid to at least one of the one or more of the bores in the cartridge; and
one or more sensors capable of measuring or otherwise detecting one or more parameters of the gas sample within the conduit.

19. The apparatus of claim 18 wherein the baffle is at least one of: configurable between a first position that directs fluid to a first subset of the one or more bores in the cartridge, and a second position that directs fluid to a second subset of the one or more bores; and shaped to direct fluid to a selectable subset of the one or more of the bores in the cartridge.

20. The apparatus of claim 18 comprising a processor, the processor connected to receive one or more signals from the one or more sensors, configured to determine a portion of the gas sample to be sampled based at least in part on the one or more signals and connected to actuate a three-way valve between a first configuration where gas is permitted to flow between the gas sample and the cartridge through the conduit and a second configuration where gas from the gas sample is prevented from reaching the cartridge.

21. The apparatus of claim 18 further comprising a detent mechanism configured to retain the cartridge in a position relative to the conduit that aligns the baffle with one or more of the one or more bores in the cartridge.

22. A method of collecting analyte from a gas sample, the method comprising:
attaching a first end of a conduit to the gas sample;
attaching a second end of the conduit to a cartridge so as to provide fluid communication between the gas sample and the cartridge through the conduit;
drawing at least a portion of the gas sample through one or more bores in the cartridge with a pressure gradient, wherein the bores are filled with sample media;
adsorbing analyte in the gas sample onto the sample media; and
measuring one or more parameters of the gas sample in the conduit with one or more sensors.

23. The method of claim 22 further comprising:
analyzing at least the one or more parameters of the gas sample within the conduit with a processor to determine a portion of the gas sample to be captured; and
actuating a valve between:
a first configuration where gas is permitted to flow between the gas sample and the cartridge through the conduit; and
a second configuration where gas is diverted before reaching the cartridge;
such that the portion of the gas sample to be captured is permitted to flow between the gas sample and the cartridge through the conduit.

24. The method of claim 23 further comprising analyzing the one or more parameters of the gas sample to determine when one or more of: (a) a concentration of a compound has been reached, or (b) when a rate of change of concentration of a compound has been reached.

25. The method of claim 23 further comprising actuating the valve from the first configuration to the second configuration when the gas sample to be captured has flowed through the sample media in the cartridge when one or more of the following are fulfilled: a rate of change of a compound is reached; a time interval has passed since the valve actuated from the second configuration to the first configuration; and when a volumetric flow rate is reached.

26. The method of claim 22 further comprising:
sensing a flow rate of gas in the cartridge with one or more sensors; and
indicating whether a particular flow rate has been achieved based at least on the measurement of the one or more sensors.
